(19) United States
(12) Patent Application Publication
(10) Pub. No.: US 2021/0282701 A1
(43) Pub. Date: Sep. 16, 2021

(54) METHOD OF EARLY DETECTION OF EPILEPTIC SEIZURES THROUGH SCALP EEG MONITORING

(71) Applicant: nCefalon Corporation, San Jose, CA (US)

(72) Inventors: Caleb K. Chan, Daly City, CA (US); An Chan, San Jose, CA (US); Jeffrey M. Chung, Los Angeles, CA (US); Li Xu, Paris (FR); Frank K. Tam, San Jose, CA (US)

(21) Appl. No.: 17/195,328

(22) Filed: Mar. 8, 2021

Related U.S. Application Data
(60) Provisional application No. 62/990,319, filed on Mar. 16, 2020.

(51) Int. Cl. A61B 5/00; A61B 5/291; A61B 5/369

(52) U.S. Cl. CPC A61B 5/4094; A61B 5/291; A61B 5/7267; A61B 5/0006; A61B 5/7225; A61B 5/369

(57) ABSTRACT

A system performs concurrent detection and early detection of epileptic seizure episodes, based on scalp electroencephalogram (EEG) of a patient collected through a data acquisition device in the course of the patient's normal daily activities. An early detection model, which is trained and retrained applying machine learning techniques at predetermined intervals on the collected data, enables issuing of an early warning of an upcoming seizure episode to allow the patient to take necessary preparatory actions (e.g., seeking a safe location for the episode to happen and alerting care-givers).

### CROSS REFERENCE TO RELATED APPLICATIONS

The present application relates to and claims priority of U.S. provisional patent application (“Provisional Application”), Ser. No. 62/990,319, entitled “A Method of Early Detection of Epileptic Seizures through Scalp EEG Monitoring, filed on Mar. 16, 2020. The disclosure of the Provisional Application is hereby incorporated by reference in its entirety.

### BACKGROUND OF THE INVENTION

### 1. Field of the Invention

The present invention relates to epileptic seizure detection. In particular, the present invention relates to (i) various aspects of detecting epileptic seizures, including early detection, and (ii) making use of a user's day-to-day scalp electroencephalogram (EEG) data to train and to continually refine a deep neural network-based model for detecting ongoing and future seizure episodes.

### 2. Discussion of the Related Art

Epilepsy is characterized by recurrent seizures resulting from chronic structural and functional changes in the brain. The unpredictable nature of seizures, i.e., not knowing when and where it would happen, causes inherent dangers to patients and profoundly degrades their quality of life. For almost a half century, evaluation of EEG is mainly carried out by visual inspection by physicians and neurologists. More recently, active research work on a patient's EEG explores the use of computer programs to predict seizures. Some examples of such work include: (i) “Seizure prediction for therapeutic devices: A review” (“Gadhoumi”), by K. Gadhoumi et al., published in J. Neurosci. Methods, vol. 260, no. 029, pp. 270-282, 2016; (ii) “A forward-looking review of seizure prediction” (“Freestone”), by D. R. Freestone et al. published in Curr. Opin. Neurol., vol. 30, no. 2, pp. 167-173, 2017; and (iii) “Prediction of seizure likelihood with a long-term, implanted seizure advisory system in patients with drug-resistant epilepsy: A first-in-man study” (“Cook”) by M. J. Cook et al., published in Lancet Neurol., vol. 12, no. 6, pp. 563-571, 2013. However, the results of the research work were mixed. Furthermore, most of them (e.g., Gadhoumi and Freestone, above) were performed in special environments (e.g., a laboratory or a hospital), so that their methods are not readily adaptable for a patient's daily use.

Some attempts have been made in the industry to make a portable device aimed at day-to-day use by a patient that provides seizure warning and allows intervention. For example, Cook reports a NeuroVista Seizure Advisory System (SAS) with electrodes, lead assemblies and a telemetry unit implanted in a patient's body. The intracranial EEG (iEEG) data measured by the electrodes was sent to a handheld device through the implanted telemetry unit. The handheld device analyzed the collected data and raised warnings, when the system expects a seizure to happen. While Cook reports results of good prediction in some patients, in other patients the prediction accuracy was low. See, for example, “Epilepsyecosystem.org: Crowd-sourcing reproducible seizure prediction with long-term human intracranial EEG” (“Kuhlmann”), by L. Kuhlmann et al., published in Brain, vol. 141, no. 9, pp. 2619-2630, 2018.

As another example, the Responsive Neurostimulator System (RNS) from NeuroPace, Inc. has electrode leads implanted in seizure onset zones in the brain to monitor the local intracranial EEG (iEEG). When abnormal iEEG signals that indicate a seizure are detected, the RNS issues brief pulses of stimulations to the brain area within milliseconds to ease or even to stop the onset of seizure before symptoms are evident. The accuracy of the RNS's seizure early detection depends on whether the intracranial electrodes are correctly placed in the seizure onset zones. In addition to direct stimulation to abort seizure at the time of electrographic onset, RNS also repeatedly administers stimulations on seizure onset zones to modulate the epileptic network and to decrease episodes of seizure. Similar intervention devices for reducing episodes of seizure also include the Vagus Nerve Stimulator (VNS) from Livallova PLC. In a regular mode, the VNS administers mild pulses to the vagus nerve periodically, 24 hours a day. In that mode of operation, the VNS does not detect whether a seizure is happening or is about to happen. The VNS may also issue, automatically or by manual intervention, an extra dose of pulses when it infers a seizure onset from a sudden increase in heart rate (e.g., a 30% to 40% increase over the resting heart rate). Unlike the RNS, the VNS does not monitor EEG for administering its stimulations. More importantly, as in SAS, these intervention systems require implanting devices and electrode leads into the patient's body, the brain, or both. Such implantations are costly invasive surgical procedures that may lead to various undesirable side effects and serious complications.

Furthermore, for medical diagnosis and evaluation purposes, the available scalp EEG data of epilepsy patients kept in the medical records most likely are biased, as the scalp EEG data are often taken under medication and medication adjustments aimed at, for example, initiating a seizure for a pre-surgical evaluation. Thus, such EEG data are not representative of the user's day-to-day life. In the previous work, any seizure prediction or early detection model trained using such EEG data would also be biased and could not predict the arrivals of seizure accurately in a patient's day-to-day life.

### SUMMARY

Unlike the previous work, a system according to embodiments of the present invention provides early seizure detections and warnings based on non-invasive measurements of a patient's scalp EEG. The non-invasive nature of the measurements allows them to be taken during the patient's regular, day-to-day life, with low cost and low risks, thereby resulting in an unbiased seizure early detection model.

According to one embodiment of the present invention, a system for seizure early detection may include (a) an acquisition device configured to be worn on a patient's head while performing every-day activities, wherein the acquisition device includes electrodes to be positioned at predetermined positions on the patient's scalp for sensing scalp electroencephalogram (EEG) data; and (b) a mobile device, wherein the mobile device receives the sensed scalp EEG data and provides the sensed scalp EEG data for (i) detecting of an ongoing seizure or (ii) determining a likelihood of occurrence of an upcoming seizure, during the acquisition device's operation, and wherein the likelihood of the upcoming seizure is determined based on a seizure early detection model trained by machine-learning techniques using the sensed EGG data currently collected and in the past. The mobile device applies the seizure detection model on the sensed EEG data. The acquisition device may include a transceiver that provides the sensed EEG data over to the mobile device over a wireless connection.

According to one embodiment of the present invention, the mobile device provides the sensed EEG data to a remote computing system on which training of the seizure early detection model takes place over a wide-area computer or communication network. The mobile device may access the wide-area computer or communication network over WiFi, or over a cellular telephone network. The remote computer system implements the machine-learning techniques in a deep neural network, such as one that long short term memory (LSTM) components. The remote computing system may include a collection of distributed computing resources and may provide each patient a virtual separate private cloud to prevent unauthorized access to the sensed EEG data and to preserve privacy.

According to one embodiment of the present invention, the remote computing system divides the sensed scalp EEG data into data segments of predetermined duration and associates each data segment to a clinical state related to epileptic seizure, as the scalp EEG data is received into the remote computing system. Each data segment may have a duration that is less than the patient's average epileptic episode, and the clinical state may be one of: “interictal-cluster,” “ictal,” “ictal-cluster,” “postictal,” “preictal,” or “interictal.” The remote computing system trains the seizure early detection model using different approaches based on performance evaluation of the seizure early detection model.

In one embodiment, during a first phase of operation, the remote computing system provides a basic seizure early detection model using publicly available scalp EEG data relevant to the patient's epilepsy type and the patient's own scalp EEG data collected under non-everyday conditions. The scalp EEG data collected under non-everyday conditions is retrieved from previous medical records. The basic seizure early detection model incorporates, in one or more calibration steps, scalp EEG data collected during performing a predetermined movement or possessing a predetermined state of mind. The predetermined movement may include one or more of: blinking of the eye, shaking and turning the head left and right, nodding and moving the head up and down, opening and closing the mouth, and smiling. The predetermined state of mind may include one or more of: closing one or both eyes to engender a relaxed mental state, focusing on one or more specific objects, and having one or more specific thoughts to engender related emotions. The remote computing system trains and retrains successively improved versions of the basic seizure early detection model until a first set of predetermined performance criteria are met, thereby providing a refined seizure early detection model, whereupon a second phase of operation is entered. During the second phase of operation, the remote computing system trains and retrains successively improved versions of the refined seizure early detection model until a second set of predetermined performance criteria are met, thereby providing a refined seizure early detection model, whereupon a third phase of operation is entered. The basic and refined seizure early detection models used in the first, second and third phases of operation are trained and retrained at different respective frequencies. The first set of performance criteria or the second set of performance criteria relate to sensitivity and specificity of the seizure early detection model to received scalp EEG data collected during occurrence of an Early state of epileptic seizure. The computing system derives a duration of the Early state. The computing system derives the duration of the Early state by applying a plurality of postulated durations on multiple postulated seizure early detection models, each created based on one of the postulated durations, and selecting at least one from among the postulated seizure early detection models as the seizure early detection model to be use in determining the likelihood of an upcoming seizure episode. The postulated durations for the Early state ranges from less than one minute to longer than one hour. If none of the postulated seizure early detection models meet a third set of performance criteria, the postulated seizure early detection model associated with the least postulated duration is selected as the seizure early detection model to be use in determining the likelihood of an upcoming seizure episode.

The present invention is better understood upon consideration of the present invention in conjunction with the accompanying drawings.

### BRIEF DESCRIPTIONS OF THE DRAWINGS

FIG. 1a illustrates an exemplary overview of a seizure detection system for early detection of epileptic seizures, according to one embodiment of the present invention.

FIG. 1b illustrates an exemplary cloud backend for the seizure detection, according to one embodiment of the present invention.

FIG. 2 is a flow chart that provides an illustrative overview of the operations of the seizure early detection system, according to one embodiment of the present invention.

FIG. 3 illustrates an immediate warning sent to a mobile device.

FIG. 4 illustrates an early “high risk of seizure” warning message sent to a mobile device.

FIG. 5a is a flow chart illustrating one procedure for scalp EEG data annotation, according to one embodiment of the present invention.

FIG. 5b illustrates the annotations of “pre-ictal,” “interictal,” “interictal-cluster,” “ictal,” “ictal-cluster,” “post-ictal,” “postictal,” and “interictal” labels on different instances of current data segment 599, relative to ictal and non-ictal segments preceding and following in time.

FIG. 6 is a flow chart illustrating a procedure for determining the duration of the Early State and the annotations of preictal data, according to one embodiment of the present invention.

FIG. 7 is a flow chart illustrating a procedure in the seizure early detection system for filtering and selecting the collected data for training and retraining. according to one embodiment of the present invention.

FIG. 8 is a flow chart illustrating a procedure for setting up, calibrating and initial operating an acquisition device, at a patient's first use of the seizure early detection system, according to one embodiment of the present invention.

FIG. 9 is a block diagram illustrating the operations of a seizure detection system that is driven by a seizure detection machine-learned model designed according to one embodiment of the present invention.

FIG. 10 shows a reduced set of electrodes—indicated by bold circles superimposed over the International 10-20 system—used in a seizure detection system in accordance with one embodiment of the present invention.

### DETAILED DESCRIPTION OF THE PREFERRED EMBODIMENTS

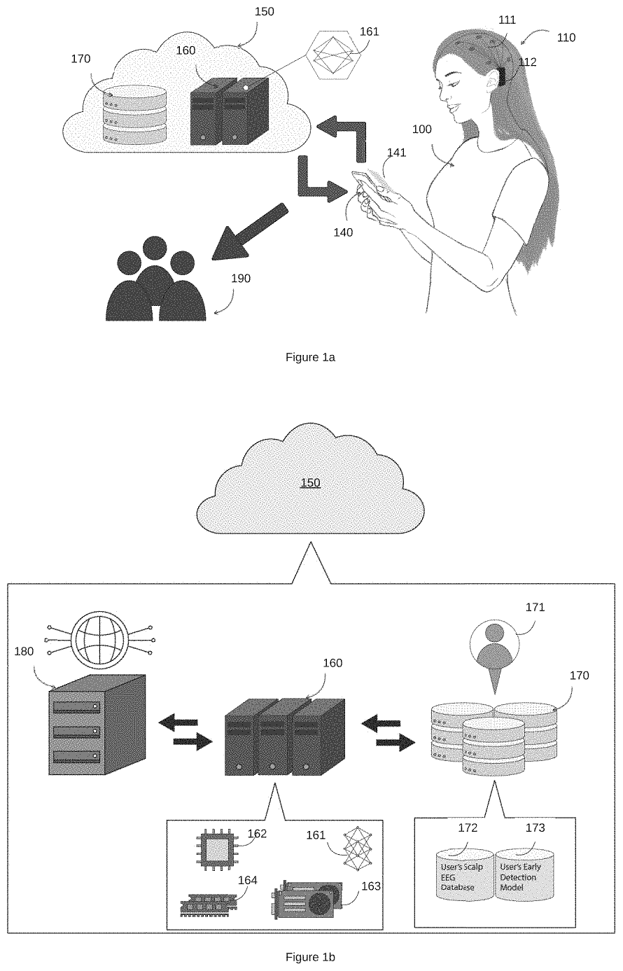

According to one embodiment of the present invention, FIG. 1a illustrates a seizure early detection system, which includes (i) scalp EEG data acquisition device 110 (“acquisition device 110”), which is worn by patient or user 100 on the head and which continuously collects the patient's scalp EEG data; (ii) mobile device 140 (e.g., a cellular “smartphone”), which patient 100 carries when wearing acquisition device 110, and which acts as a user interface for patient 100 to interact with acquisition device 110 and backend system 150. In one embodiment, mobile device 140 also streams the scalp EEG data collected by acquisition device 110 in real time to backend system 150 for processing; and (iii) backend system 150, which is a set of networked computing and storage devices, receiving and processing the scalp EEG data streamed from mobile device 140 or directly from acquisition device 110.

Acquisition device 110 includes (i) multiple electrodes 111, which are positioned to contact patient 100's scalp, so as to collect scalp EEG data, and (ii) wireless transceiver 112 that communicates the collected scalp EEG data to a receiving device. In the example of FIG. 1a, the scalp EEG data is transmitted from wireless transceiver 112 on acquisition device 110 to mobile device 140. In turn, mobile device 140 relays the received scalp EEG data to backend system 150 for further processing and storage. Backend system 150 is also referred to as a “cloud backend,” as it represents a collection of distributed or networked computing resources operating the seizure early detection system that reside in a wide-area computer network (e.g., the “internet”). Within acquisition device 110, a cyclic buffer temporarily stores the collected scalp EEG data until it is transmitted by wireless communication (e.g., Bluetooth or WiFi) from acquisition device 110 to mobile device 140. In one embodiment, acquisition device 110 merely continuously collects and transmits patient 100's scalp EEG data and does not process or analyze the collected scalp EEG data. Mobile device 140's memory and storage (e.g., a cyclic buffer may be allocated in mobile device 140's memory) temporarily store the scalp EEG data until it is communicated to backend system 150 over the wide-area computer network. Mobile device 140 may access the wide-area computer network over WiFi or a cellular communication network.

At backend system 150, processing unit 160 maintains a deep neural network-based model set (“model set”) 161. In the context of this detailed description, a model set refers to one or more trained deep neural network models that process and analyze scalp EEG data. In processing unit 160, model set 161 continuously infers from patient 100's scalp EEG data in real time whether a seizure episode may happen to patient 100 within a certain future time frame. If model set 161 infers that a seizure is expected, an alert or warning message is sent to mobile device 140 to alert patient 100. The warning message may take any suitable form, such as device vibration, an audio alarm (i.e., sound), a pop-up message, a voice message, a telephone call, or any suitable combination of these modalities. Patient 100 may also set up in advance to have alert or warning messages sent concurrently to other people, indicated in FIG. 1a as alerted parties 190. Alerted parties 190 may include caregivers (e.g., nurses or physicians). Warning messages sent to alerted parties 190 may be the same as or different from the warning message received by patient 100.

The warning message sent from backend system 150 enables patient 100 to make preparations ahead of the epileptic episode, such as suspending the current activity (e.g., walking), seeking and retreating to a safe place, and sitting down at the safe place, so as to avoid falling down during the seizure, when patient 100 is likely unconscious. Also, the warning messages properly inform and enable alerted parties 190 to take possible actions to assist patient 100. Significantly, also, the processed scalp EEG data is processed and retained in storage 170 to allow further analysis and for training and updating the models in model set 161.

Patient 100 communicates with the rest of the seizure early detection system on backend system 150 via interactions with application program 141 on mobile device 140. Application program 141 runs on mobile device 140 and provides a graphical user interface (GUI) to facilitate the interactions, which may include system configuration and control, and communicating with (e.g., exchanging messages) the seizure early detection system. System configuration and control may include starting scalp EEG monitoring, setting up the form of the warning messages, identifying the intended alerted parties to the seizure early detection system, and sending feedback to the system, and any suitable control or configuration activities. In addition to the warning messages from backend system 150 which alert patient 100 when a seizure is expected to happen, patient 100 may receive from the seizure early detection system messages that report errors in operation and other suitable information. As mentioned above, mobile device 140 may be a third-party device (e.g., a smart phone associated with a cellular service provider), or a custom device designed specifically for the seizure early detection system.

FIG. 1b shows in greater detail backend system 150, according to one embodiment of the present invention. As shown in FIG. 1b, backend system 150 includes: (i) networking unit 180; (ii) processing unit 160 and (iii) storage unit 170. Once patient 100 signs up for an early seizure detection service, a “virtual separate private” cloud is created under patient 100's account (e.g., user account 171). The virtual separate private cloud represents a set of remote, networked computing resources so securely configured to prevent unauthorized access by third parties and to protect patient 100's privacy that the remote, networked computing resources appear as if they are dedicated to the patient's exclusive use.

Networking unit 180 handles the computer network connections between backend system 150 and the rest of the seizure early detection system. In our embodiment, backend system 150 receives user commands and data from mobile device 140 and sends alert and commands to mobile device 140. In other variations, e.g., through WiFi or cellular networks, backend system 150 may receive EEG data directly from acquisition device 110; and may also directly send alert or warning messages to communication devices of alerted parties 190 through networking unit 180.

Processing unit 160 performs: (i) detection, (ii) model training and (iii) general data processing. In one implementation, processing units may include general purpose central processing units (CPUs) 162, graphics processing unit (GPU) farm 163 and memory 164. In this embodiment, model set 161 is trained and run in the large number of GPUs available on GPU farm 163. Model set 161 takes the scalp EEG data received in real time to infer if the data indicates that patient 100 is experiencing or about to experience a seizure. The models in model set 161 may be trained or retrained in the same set up on GPU farm 163, using training and testing data from storage unit 170. When retraining happens as designed or scheduled (as discussed in further detail below), or when desired, GPU farm 163 retrains the early detection models based on the collected scalp EEG data, or other suitable data, to update model set 161. Updated model set 161 is then implemented to process future received scalp EEG data. Model set 161 is maintained and archived in storage unit 170. Processing unit 160 also performs other general data processing, e.g., calculating statistical evaluation metrics for different versions of detection models, annotating and tagging timestamps to data segments collected, and other suitable activities.

Storage unit 170 maintains user account 171, patient 100's scalp EEG database 172 and patient 100's early detection model zoo 173. User account 171 includes patient 100's epilepsy medical records, user-supplied information and account-related information. Data maintained in account 171 is encrypted and complies with the requirements of the Health Insurance Portability and Accountability Act of 1996 (HIPAA). The records in scalp EEG database 172 have been processed to include tagging timestamps, annotations and filters (which are described below). Early detection model zoo 173 is a database that maintains at least one copy of current model set 161, the most recently retrained early detection models and, optionally, copies of other versions of the retrained early detection models. These other versions of early detection models are maintained primarily for the seizure early detection system's internal use (e.g., to evaluate performance metrics of the models and for future retraining). The data in scalp EEG database 172 and early detection model zoo 173 refer to patient 100 only to a user identification number (“user ID”). Thus, unless one can associate the user ID with the corresponding user account 171, scalp EEG database 172 and early detection model zoo 173 are for all purposes anonymous.

All patient's data (e.g., EEG data) is encrypted for the protection of data confidentiality and integrity. Encryption is used not only for data stored in backend system 150, but also for transmitted to or retrieved from backend system 150. Examples of the encryption algorithms that may be used include AES256 (Advanced Encryption Standard-256-bit key).

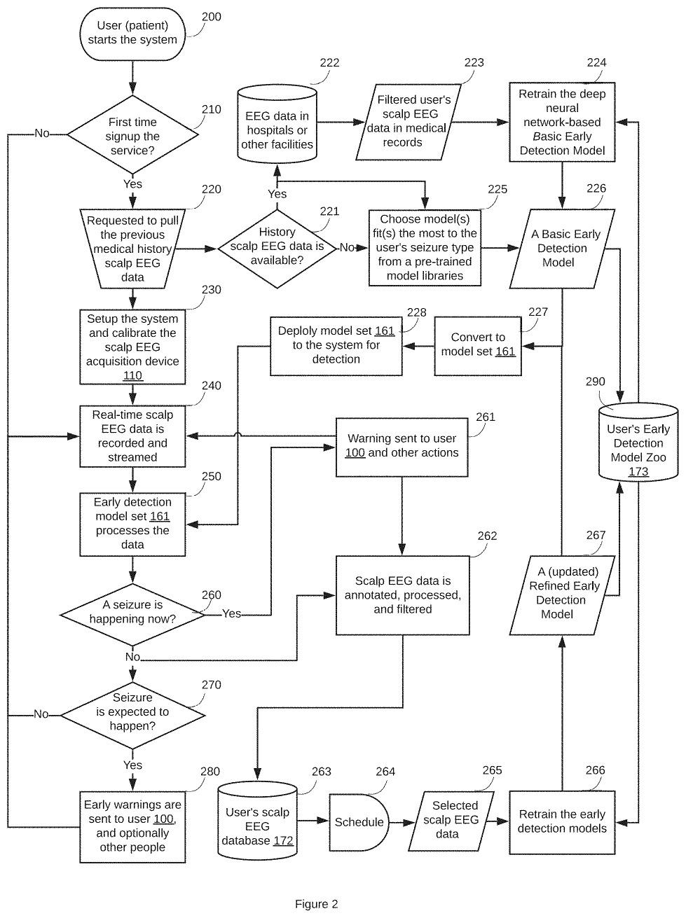

FIG. 2 is a flow chart that provides an illustrative overview of the operations of the seizure early detection system, according to one embodiment of the present invention. As shown in FIG. 2, initially, patient 100 places the scalp EEG acquisition device 110 on the head and begins system operation using application program 141 that runs on mobile device 140. At patient 100's first use of the seizure early detection system (i.e., at decision step 210), steps 220 and 230 configure the system for subsequent uses. As background, patient 100—who presumably has been previously diagnosed with epilepsy—is expected to have some basic information (e.g., the type of seizure experienced), and to have, possibly, previously measured scalp EEG data in his or her medical records.

Thus, at step 220, patient 100 is asked about and to request for, if available, medical records from the care givers, including previously collected scalp EEG data. Whether or not patient 100 has previously collected scalp EEG data, at step 225, the seizure early detection system creates a patient profile for patient 100 based on a previously trained model library using patient-supplied information, such as the type of seizure experienced, age group and gender. The trained model library has been built from (i) publicly available scalp EEG data pooled from different anonymous patients; and (ii) scalp EEG data from other patients of the seizure early detection system that have been processed to ensure anonymity.

Within the seizure early detection system, the pooled anonymous scalp EEG data is grouped according to seizure types, ages and genders, and any other suitable relevant information categories. Each group of data is then used to train one or more early detection models. As these models are trained with EEG data pooled from multiple anonymous patients and users, who are not related to patient 100, there may be more than one possible trained model candidate for representing patient 100's seizure type, as no one trained model may exactly fit patient 100's seizure type. The collection of selected trained models forms patient 100's “Basic Early Detection Model” or model set 226, which becomes part of patient 100's early detection model zoo 173 at step 290. Model set 226 is converted to model set 161 at step 227. Model set 161 then becomes available for process unit 160 to perform basic early seizure detection (“prediction”), as indicated at step 228.

Model set 226 and model set 161 include substantively the same information, except that, for deployment purposes, some conversions may be required. In this description, the deployed model set is designated model set 161. Model set 226 is a “Basic Early Detection Model” because it is not sufficiently trained to patient 100's day-to-day scalp EEG patterns. Even so, model 226 makes possible early detection of seizure before a significant amount of patient 100's own scalp EEG data has been incorporated.

If patient 100's previous scalp EEG data is available, the data is obtained from where the records are kept (e.g., the records represented in FIG. 2 by database 222) and is uploaded to backend system 150 into patient 100's virtual private separate cloud. At step 223, processing unit 160 filters the uploaded data. For example, based on patient 100's medical records, processing unit 160 may select only the EEG data collected from channels close to the seizure focus and may discard other measurements, when appropriate. Processing unit 160 may perform additional necessary preprocessing (e.g., frequency filtering). At step 224, the filtered data is then used to retrain model set 226, which is then incorporated into models in early detection model zoo 173. Retrained model set 226, which replaces models selected from the previously trained model library, has higher accuracy with respect to detecting and predicting patient 100's seizures, as model set 226 has appropriately incorporated user-specific scalp EEG data in the medical records. At this point, as the data that is used to create model set 226 is primarily collected under special environments (e.g., hospital and medicated conditions), the data inherently includes biases, as already explained in with respect to the prior art data discussed above. To eliminate the biases, the seizure early detection system of the present invention continuously fine-tunes model set 226 based on patient 100's own day-to-day EEG data.

At step 230, through application program 141 on the mobile device 140, patient 100 customizes the seizure early detection system and calibrates acquisition device 110, as discussed in further detail below. At step 240, acquisition device 110 initiates scalp EEG data collection and mobile device 140 begins to stream the data to backend system 150.

When model set 161 (i.e., the deployed model set) includes more than one early detection model, each model may have a different duration for the “Early State” (discussed below), trained characteristics of a different seizure type, or both. The early detection models infer from the data in parallel to provide more reliable detection results. Upon collecting contemporaneous scalp EEG data at step 250, model set 161 (i) detects in real-time whether or not patient 100 is currently experiencing a seizure at decision step 260 and (ii) if not, infers a probability of a seizure to occur within a certain future time window at decision step 270.

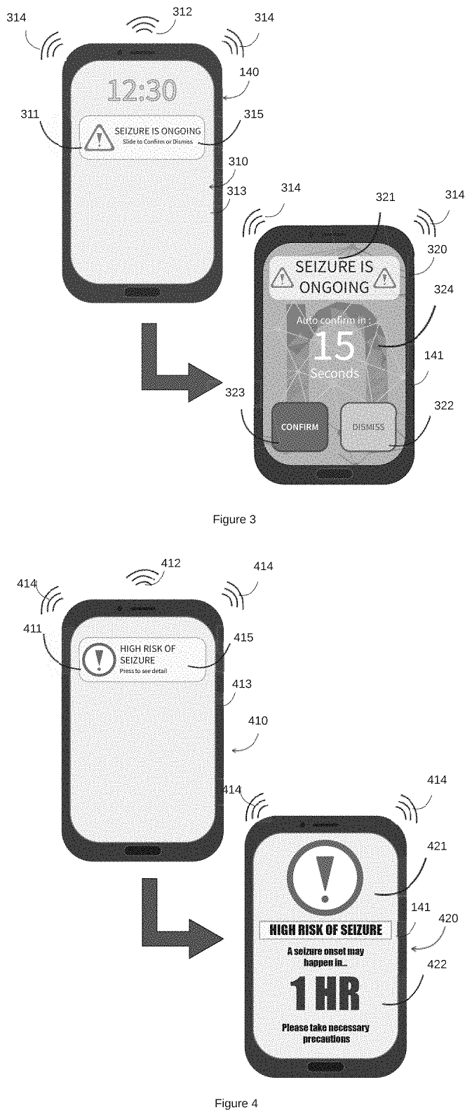

If it is determined that patient 100 is experiencing a seizure at decision 260, an immediate warning message is sent to patient 100's mobile device 140 at step 261 and the contemporaneous EEG data are annotated, processed and filtered at step 262. FIG. 3 illustrates an immediate warning that is sent to mobile device 140. As shown in FIG. 3, if the immediate warning message arrives at mobile device 140 at a time when application program 141 is not open, notification message 311 appears on screen 310 on top of any open application program displayed on application screen 313, a screen-saver, or any other screen background. As shown in FIG. 3, notification message 311 indicates that a seizure episode is ongoing and asks patient 100 to use a sliding gesture to “confirm” the existence of an ongoing seizure, or “dismiss” (as shown in text 315) the notification message. Mobile device 140 may also provide vibrations 314 and to make continuous “beep” sounds 312 to amplify the message.

Alternatively, if application program 141 is open at the time the immediate warning message arrives, or if patient 100 has responded to warning message 311 (e.g., by performing the sliding gesture), screen 320 provides options for patient 100 to confirm that he or she is experiencing a seizure or to dismiss the warning message as being a false alarm. Meanwhile, mobile device 140 continues to vibrate. If no response is returned from patient 100 after 30 seconds (or any suitable time period, selectable by patient 100), the seizure early detection system deems patient 100 to have become too decapacitated to respond and therefore confirms the seizure onset without user input. Timer widget 324 shows the time remaining for patient 100 to perform the confirmation or dismissal action. Patient 100 may specify in the settings that to have a loud alarm to go off to alert people in the surrounding, or to have a message or a voice call be made to patient 100's caregivers, or to have another suitable action taken. FIG. 3 is merely exemplary, any other suitable GUI or operation of application program 141 may be used. At step 263, the annotated, processed and filtered scalp EEG data is stored into database 172.

If a high probability of seizure in the immediate future is inferred, processing unit 160 sends an early warning message to patient 100 over mobile device 140 (step 280). Other suitable actions, such as alerting patient 100's caregivers, may also be taken. Throughout this time, real-time collection and streaming of scalp EEG data continue (i.e., at step 240).

At an appropriate time, based on schedule 264, selected scalp EEG data 265 is extracted from database 172 and retraining of the deep neural network-based models is performed at step 266. The targeted retrained model set may be, for example, model set 226, unless a model set already exists that is trained or retrained using patient 100's own day-to-day scalp EEG (referred herein as “Refined Early Detection Model,” or model set 267). After the requisite model or models in model set 267 are retrained and model set 267 is updated. The model set may be retrained from model set 226 or 267, the retrained model set is referred to as model set 267. Model set 267 is then stored in model zoo 173. Model set 267 is referred to as “Refined” because of its improved accuracy due to its training using patient-specific day-to-day scalp EEG data. Model set 267 also replaces model set 226 in model set 161, after suitable conversions, to become the current deployed model set residing in backend system 150. Schedule 264 may be set up to retrain and to regenerate model set 267 at suitable intervals, as described in further details below, as more and more day-to-day scalp EEG data is collected from patient 100.

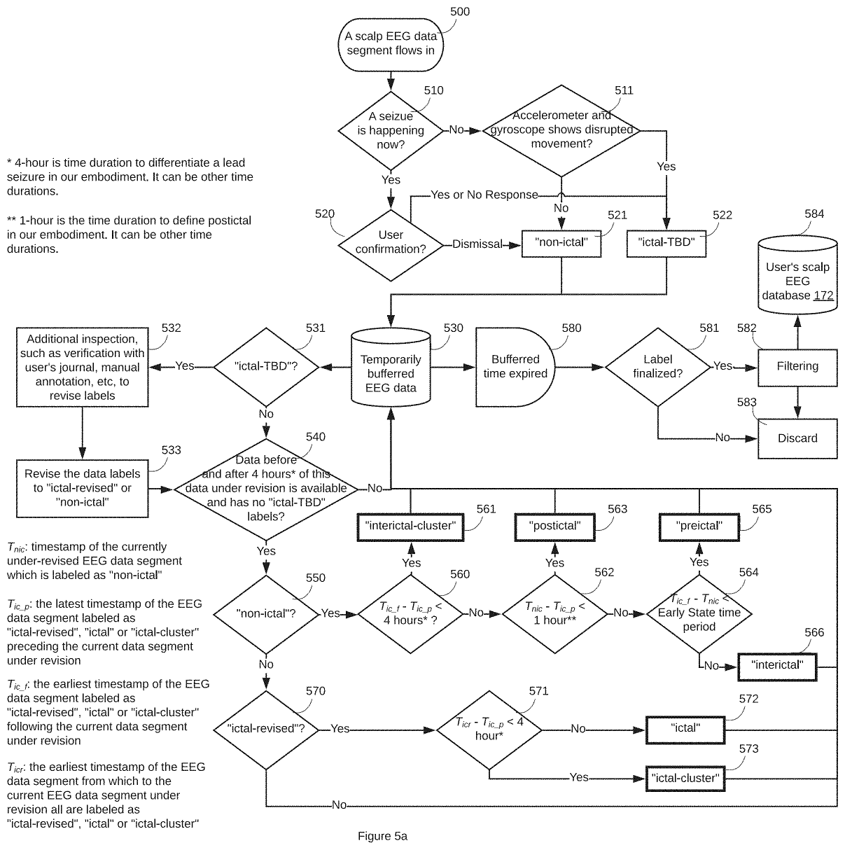

As mentioned above, at decision step 270, a probability of seizure happening in a predetermined immediate future time window is inferred. In the seizure early detection system, a threshold (“high risk of seizure” threshold) is set, which triggers an early warning message to patient 100 through the mobile device 140, at step 280. FIG. 4 illustrates an exemplary “high risk of seizure” early warning message that is sent to mobile device 140. If application program 141 is not open in mobile device 140 at the time when early warning message 411 is sent, early warning message 411 may be shown superimposed on top of screen 413, which may be the screen displayed for another application program, a screen-saver, or any other screen background. Message 411 warns of a high risk of seizure in an immediate future time window and invites patient 100 to tap text box 415 to access the details. Mobile device 140 may also vibrate at step 414 and may provide an alerting “beep” sound at step 412, if so set. Screen 420 is displayed if application program 141 is open, or if patient 100 has tapped warning message 411 as directed. In screen 420, mobile device 140 continues to vibrate and message 421, warning of a high risk of seizure, is shown together with expected seizure onset time 422.

In the example of FIG. 4, expected seizure onset time 422 is one hour, which is a probabilistic expectation value, not a prediction that a seizure will happen in exactly one hour. Expected seizure onset time 422 communicates that a high probability of a seizure happening exists within a future time window expected to be one hour and serves as an early warning to enable patient 100 to take necessary precautions before any actual seizure onset happens. The value of the expected seizure onset time depends on the early detection model in current use, the scalp EEG data segment received and the adopted definition of the duration of the Early State (further discussed below). Accordingly, expected seizure onset time 422 may vary from patient to patient, and may even vary for the same patient over time. The seizure early detection system may take various actions, based on patient 100's system setting. For example, messages or voice calls may be sent or made to patient 100's caregivers.

Rather than deploying model set 161 on backend system 150, as described above, model set 161 may also be deployed on mobile device 140, to allow mobile device 140 to perform early detection. Under that option, backend system 150 performs only scalp EEG data annotations, storage and model training or retraining, but early seizure detection warnings would be available to patient 100 even if a data network connection is not.

Alternatively, local detection (i.e., having early detection performed on mobile device 140) and remote detection (i.e., having early detection performed on backend system 150) may work together in a complementary fashion. In that case, both backend system 150 and mobile device 140 each have a copy of model set 161 to perform the early detection task. As mobile device 140 is likely to have resource constraints, model set 161 on mobile device 140 may not be the most up-to-date version, or may be a simplified version, which sacrifices some detection accuracies. Under normal operating conditions, remote detection is available to detect ongoing and incoming seizures. However, when the data network is not available, local detection provides a fall-back position to avoid service interruption.

Model set 161 need not be the only deployed model set. More than one model set may be deployed simultaneously. For example, a Seizure Detection Model may be deployed for detecting an ongoing seizure, while an Early Seizure Detection Model detects a probability of a seizure taking place in an immediate future time window. In that case, one of the two models may be deployed on mobile device 140, while the other model is deployed in backend system 150, for example. Of course, it may be possible to have both model sets deployed on mobile device 140, or both model sets deployed in backend system 150.

The Basic Early Detection Model and the Refined Early Detection Model are trained using different data, even though the underlying neural network architectures of these models are substantially the same. Accordingly, the generic term “seizure early detection models” refer to both model sets. The seizure early detection models receive as input patient 100's scalp EEG data and provide as output the epileptic state indicated by the scalp EEG data. In the seizure early detection system, the following epileptic states of scalp EEG data are used for reporting and for annotation:

1. Ictal
“Ictal” refers to the state of an ongoing lead seizure. A lead seizure is a clinical seizure which occurs at least 4 hours after the previous seizure onset. In some literature, a lead seizure is more conservatively defined as a seizure occurring at least 8 hours after the previous seizure. Therefore, the time interval may be set in the seizure early detection system, as desired.

2. Preictal
“Preictal” refers to a state before the onset of a lead seizure. When in use, the seizure early detection system determines, using statistical techniques, a state referred to as an Early State, which precedes the onset of a lead seizure. When the scalp EEG data has a timestamp within the Early State, the scalp EEG data is annotated “preictal,” as discussed below.

3. Postictal
“Postictal” refers to a state within, for example, one hour after a lead seizure ends. The duration of the preictal state may be set in the seizure early detection system, as desired.

4. Interictal
“Interictal” refers to a state after a postictal state but before the next preictal state.

5. Ictal-cluster
“Ictal-cluster” refers to a state of an ongoing seizure which occurs within 4 hours after the end of a previous seizure, when lead seizures are defined to be 4 hours apart. The duration of the Ictal-cluster state may be set in the seizure early detection system, as desired.

6. Interictal-cluster
“Interictal-cluster” refers to the state between ictal-cluster states.

The seizure early detection models described herein are neural network-based models that have a Long Short-Term Memory (LSTM) deep neural network architecture. A LSTM-based network is a variation of a recurrent neural network (RNN), which is a neural network that learns from input data that is sequence-dependent over time. For an RNN, apart from being trained using input data at current time t, each training cycle also takes into account the state of the network from previous time t−1 to determine the current output or prediction. Thus, the output or prediction of the model depends on both the input data at the current and, inherently, data from all previous times.

A conventional RNN only looks at the state of the neural network from the immediately previous time point in the input data sequence, which limits the manner history or memory is used in the RNN for predicting future outputs. The LSTM technique circumvents this limitation by feeding two network states back into the network as current input, one from the previous time point, and the other from a time point that is further back in history. As a result, the output of an LSTM-based network infers current output based on explicit treatments of both long-term and short-term memories of the input data. This property significantly improves detection of ongoing and incoming seizures, as scalp EEG data is highly temporally correlated, so that inclusion of data from recent and distant time points adds useful information to seizure detection.

LSTM-based neural networks have been used in natural language processing (NPL) which, due to semantic meanings, has input data with well-defined boundaries (e.g., words, phrases and sentences). Scalp EEG data, however, does not have well-defined boundaries to guide input. Accordingly, in one embodiment of the present invention, the scalp EEG data is divided into sequences of uniform duration (“data segments”), and an output (i.e., the predicted state) is provided only after one or, alternatively, a predetermined number of sequences are completely processed in the neural network.

Other non-LSTM realizations of the seizure early detection system are possible. For example, other forms of time-independent classifiers—i.e. models—such as support vector machines (SVMs) or convolutional neural networks (CNNs) can also be used. These models, however, do not take advantage of the temporal information and require a feature selection process. In general, feature selection is a manual process, which may or may not be user-specific and time-dependent, as it involves selection of specific frequency bands or power spectra that provide the best predictive power.

According to one embodiment of the present invention, the seizure early detection system collects and annotates the scalp EEG data to facilitate training the seizure early detection models. First, when the scalp EEG data is continuously received into the seizure early detection system, they are grouped into data segments for receiving into the current early detection model or models (e.g., model set 161). Preferably, the duration of each data segment is shorter than the average duration of a seizure episode. Therefore, the appropriate duration of a data segment is user-specific, as a suitable duration may be different from patient to patient. In one embodiment, the duration of a seizure episode for each patient is determined from the patient's epilepsy medical history and the statistical average based on the collected scalp EEG data over time. A data segment duration is then set to a value shorter than the determined seizure episode duration. In the following detailed description, one minute is used as a convenient example of a suitable data segment duration. So long as the data segment duration is shorter than the determined duration of a seizure episode, the data segment duration is a design parameter that trades-off between detectability and delay. While a longer data segment duration allows the early detection models to find more patterns, it results in a longer delay for the patient to receive a detection result.

Data segmentation may be implemented with either fixed boundaries or sliding a window. For fixed boundaries, the end of each data segment is also the beginning of a next data segment, i.e., data segments do not overlap each other. For a sliding window, the sliding window slides over a data segment with a much smaller step size than the data segment size, so that two consecutive data segments overlap each other. Using a sliding window, the same duration provides a larger number of data segments as compared with using fix boundaries. While the sliding window approach has a higher computational cost for a neural network model training, the approach may also increase model accuracy, as the sliding window approach captures the evolution of EEG signals. The following description of data segments and how they are annotated apply on both fixed boundaries and sliding window implementations.

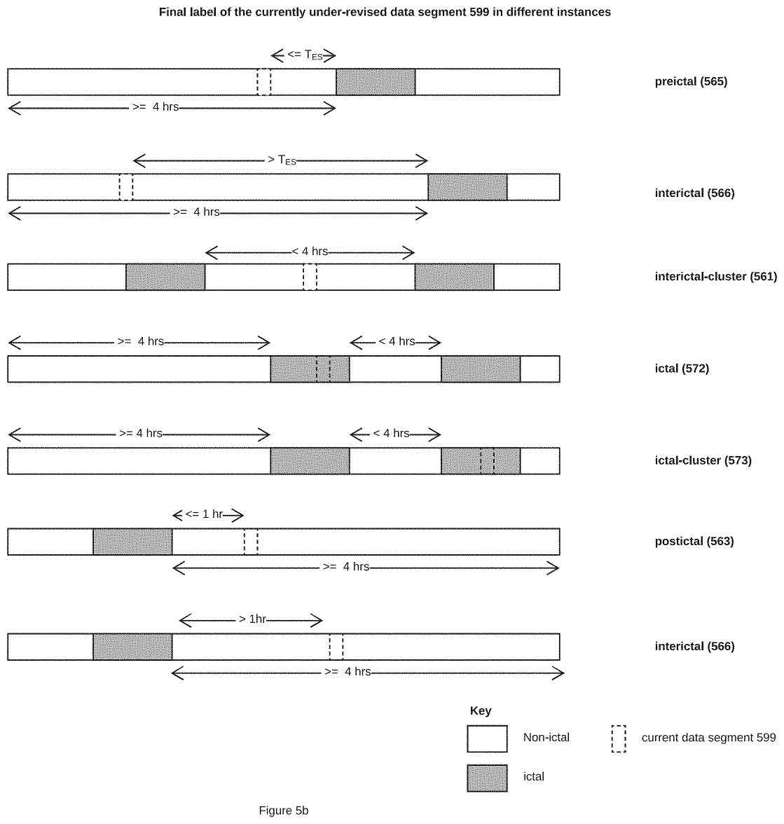

A timestamp is associated (“tagged”) with each scalp EEG data segment as it is being recorded. The timestamp may refer to the time at the mid-point of the data segment. For example, if a scalp EEG data segment starts at 13:40:00 and ends at 13:41:00 (one-minute duration), the scalp EEG data segment may be associated with a 13:40:30 timestamp. The timestamp may also include references to the year, month and date (i.e., an “epoch time”). As described above, detection of an ongoing seizure in model set 161 plays an important role in scalp EEG data annotation. FIG. 5a is a flow chart illustration one procedure for scalp EEG data annotation.

When a data segment of scalp EEG data is received into early detection model set 161 at starting point 500, model set 161 determines if the patient is experiencing a seizure. If so, at step 520, the seizure early detection system waits for a patient confirmation, as described above in conjunction with FIG. 3. When the patient dismisses the warning message sent, the data segment is labeled “non-ictal” at step 521. Otherwise, i.e., a patient confirmation is received or a patient response is absent after the preset time, the seizure early detection system deems that an ongoing seizure condition may exist and labels the scalp EEG data segment “ictal-TBD” at step 522. Neither “non-ictal” nor “ictal-TBD” is a final label in the annotated scalp EEG data; the “non-ictal” and the “ictal-TBD” labels are subject to subsequent revision or refinement.

At decision step 510, if an ongoing seizure is not detected, the seizure early detection system reads an accelerometer or a gyroscope provided in either acquisition device 110 or mobile device 140 the seizure early detection system as supplementary data, so as to avoid missing an ongoing seizure event. At decision step 511, a disrupted movement inferred from the accelerometer or gyroscope data causes the scalp EEG data segment to be labeled “ictal-TBD” at step 522; otherwise, the EEG data segment is labeled “non-ictal” at step 521. The labeled scalp EEG data segment is stored temporarily in a buffer (e.g., in database 530). Thus, accelerometer and gyroscope readings are used as probative data to help determine an ongoing seizure in real-time. If acquisition devices 110 or mobile device 140 has another seizure-sensitive recording device (e.g., a microphone) and, if such a device is active, the seizure early detection system may also be used as additional supplementary data to help determine an ongoing seizure.

The data stored in database 530 is examined on a regular basis (e.g., daily), preferably at an interval longer than twice the minimum time between consecutive lead seizures. Setting this interval involve trading-off between delay and the cost of system computational power. The seizure early detection system uses the regular examinations of database 530 (i.e., steps 531-571) to revise and finalize the labels annotated to the scalp EEG data segment.

Through decision by model set 161, or the confirmations based on supplementary data (e.g., the readings of the accelerometer or the gyroscope), a scalp EEG data segment is either labeled “ictal-TBD” or “non-ictal” at this stage. At step 531, if a data segment is labeled “ictal-TBD,” the seizure early detection system may perform additional inspections at step 532. (In some alternative implementations, even “non-ictal” data are provided an additional inspection, especially in the beginning when model set 161 has not been sufficiently retrained to achieve high accuracy for patient 100.) This detailed description discusses, for illustrative purpose only, additional inspection only with respect to data segments that are labeled “ictal-TBD.” However, the additional inspection for “non-ictal” data segments may be similar. Additional inspections may include comparing the current annotations to patient 100's journal of seizures, manual annotations, or determinations by another more sophisticated deep neural network model. (These more sophisticated neural networks, though having a higher seizure detection accuracy, may be too computationally intensive for real time processing.) As a result of the additional inspection, a data segment labeled “ictal-TBD” may be revised to either “ictal-revised” or “non-ictal” at step 533, to indicate that the additional inspection confirms that the data segment is possibly ictal data, or not actually ictal data, as the case may be.

Thereafter, at step 540, the seizure early detection system further determines if any other revised data (i.e., data with no data segment labeled “ictal-TBD”) exists that is more than four hours (i.e., the adopted minimum time between lead seizures) before or after the current revised data segment. In this example, the minimum time between lead seizures is set as four hours in the seizure early detection system for merely illustrative purpose. If not all data segments within this time period are available and have been revised, the current revised data segment is left in database 530 to be revisited in the next examination. Otherwise, at step 550, if the current revised data segment is “non-ictal,” steps 560-566 are carried out.

At step 560, the seizure early detection system searches near the timestamp of the data segment currently under revision. The results include (i) the latest EEG data segment labeled “ictal-revised”, “ictal” or “ictal-cluster” preceding the current data segment under revision, which is then assigned timestamp T_{ic_p}; and (ii) the earliest EEG data segment labeled “ictal-revised”, “ictal” or “ictal-cluster” following the current data segment under revision, which is then assigned timestamp T_{ic_f}. Seizure early detection system determines if the difference between T_{ic_j}, and T_{ic_f} is less than four hours. If so, at step 561, the current revised “non-ictal” data segment is annotated “interictal-cluster.” Otherwise, the subsequent ictal data, i.e. the data segment with the timestamp T_{ic_f}, belongs to a lead seizure.

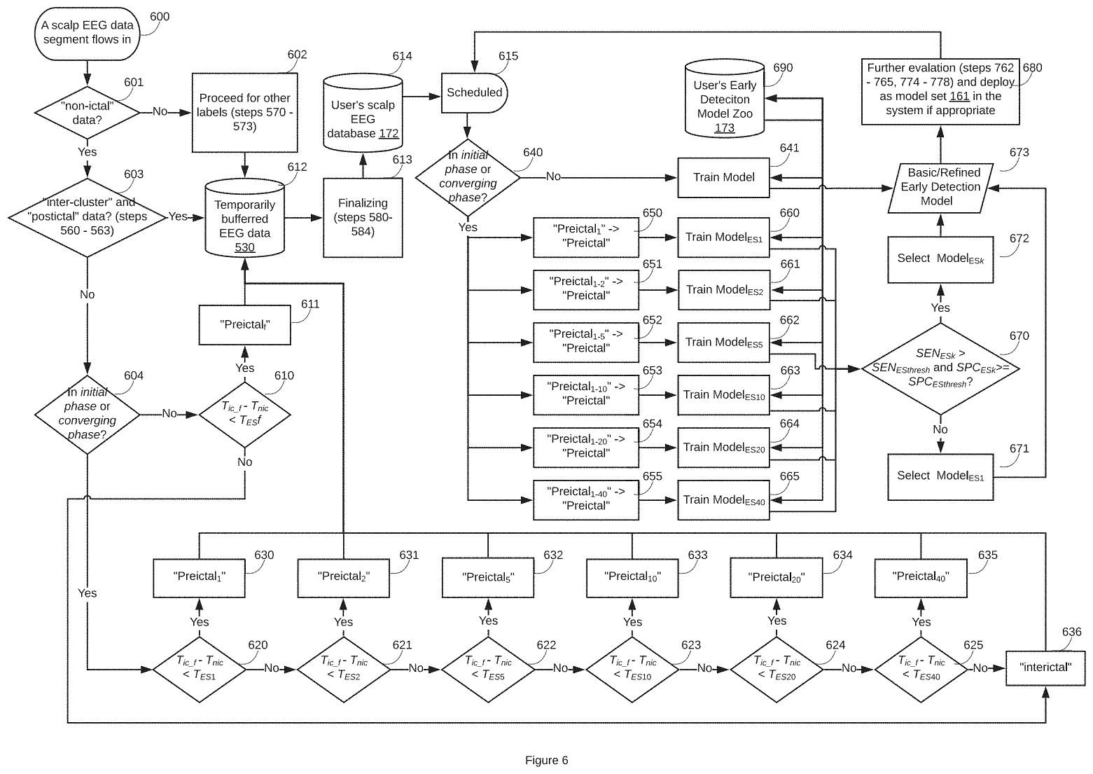

At step 562, the seizure early detection system further determines if the latest preceding ictal data is less than one hour prior to the current “non-ictal” revised data segment. If so, at step 563, the current “non-ictal” revised data is annotated “postictal.” Otherwise, at step 564, the seizure early detection system further determines if the earliest ictal data subsequent to the current “non-ictal” data segment (i.e., the next lead seizure) is within the Early State, which duration is determined, for example, according to a procedure described below in conjunction with FIG. 6. If so, at step 565, this “non-ictal” revised data segment is annotated “preictal”. The “preictal” annotation here is a simplified one, as would be apparent in the further discussion below.

At step 566, having determined in steps 560, 562 and 564 that the current “non-ictal” revised data segment is not “interictal-cluster,” “postictal,” or “preictal,” the current “non-ictal” revised data segment is annotated “interictal.”

Returning to decision step 550, if the current revised data segment is not “non-ictal,” the seizure early detection system determines, at step 570, whether it is “ictal-revised.” If so, the seizure early detection system first looks for the EEG data segment that precedes the data segment currently under revision. If the preceded data segment is labeled “ictal-revised”, “ictal” or “ictal-cluster”, the seizure early detection system continues to look at earlier data segment until it finds an earlier data segment which is not labeled as “ictal-revised”, “ictal” or “ictal-cluster”. The seizure early detection system then assigns the earliest timestamp in this sequence of ictal-labeled data segments T_{icr}. At step 571, the seizure early detection system determines if the difference between T_{icr} and T_{ic_p} is less than four hours. If so, this current “ictal-revised” data segment under revision is part of a seizure cluster. Accordingly, the current “ictal-revised” data segment is annotated “ictal-cluster” at step 573. Otherwise, i.e., the current “ictal-revised” data segment is not within four hours of a preceding ictal data, the current “ictal-revised” data segment is annotated “ictal” at step 572. Returning back to decision step 570, if the current data segment is neither “non-ictal” nor “ictal-revised,” the current data segment should already have been annotated “ictal”, “preictal”, “postictal”, “interictal”, “ictal-cluster”, or “interictal-cluster”. No further relabeling or annotating is necessary.

FIG. 5b illustrates the annotation of “pre-ictal” (step 565), “interictal” (step 566), “interictal-cluster” (step 561), “ictal” (step 572), “ictal-cluster” (step 573), “post-ictal” (step 563) labels on different instances of current data segment 599, relative to ictal (shaded) and non-ictal (blank) segments preceding and following in time.

Returning to FIG. 5a, the annotated data is returned to database 530 until buffer time 580 expires. At the expiration of buffer time 580, steps 581-584 are carried out. Buffer time 580 (e.g., two days) expires when the time difference between the current system time and the timestamp of the annotated data segment reaches buffer time 580. Buffer time 580 allows labeling of data segments in database 530 to be finalized (i.e., steps 531-573 to be carried out). Buffer time 580 is set based on the how often the regular examination of the data occurs and should be set greater than the minimum time for every data segment to be revised. In one embodiment, database 530 is examined daily, i.e. at 12:00 AM every day, when data segment, dₚ, collected between 12:00 AM to 12:01 AM of the previous day (say, timestamp Tₚ) is revised for the first time. To determine if dₚ is within a seizure cluster or not, as explained with respect to steps 531-573, the seizure early detection system requires examination of data collected 4 hours prior to Tₚ. Thus, the data collected 4 hours prior to Tₚ is retained in database 530 and should not have been removed due to expiration. Therefore, a suitable buffer time may be 28 hours or longer (e.g., two days), in one embodiment. On the other hand, a longer buffer time delays availability of the most up-to-date scalp EEG data in database 172, so that retraining of model set 161 becomes even later.

When buffer time 580 expires for a data segment, the seizure early detection system determines at step 581 if the data segment has been annotated with its final label. If so, the data segment is filtered at step 582. The filter may discard data that is deemed unreliable for artifacts in acquisition device 110 (e.g., no signal, signal amplitudes out of range). Some other data may also be discarded based on system requirements, as discussed below. After filtering at step 582, the scalp EEG data is written at step 584 into patient 100's scalp EEG database—which is referred herein as database 172—for storage and subsequent use in training and retraining of the seizure early detection models. At step 583, any data segment not annotated with a final label is also discarded.

The term “Early State” refers to a state that is prior to and terminates at the onset of a lead seizure. However, the onset of an Early State is unknown and requires using patient-specific scalp EEG data collected by the seizure early detection system to infer. In one embodiment of the present invention, the seizure early detection system has roughly three phases according to the amount of scalp EEG data collected from the patient and the quality of training of the early detection models using the collected data: (i) initial phase, (ii) converging phase, and (iii) performance phase. The initial phase generally refers to the initial time period after the patient begins to use the seizure early detection system. During the initial phase, model set 161 corresponds to the Basic Early Detection Model and patient-specific day-to-day scalp EEG data is being aggressively collected, and the early detection models are periodically retrained. The initial phase ends, and the converging phase is entered, when evaluation metrics of a newly trained or retrained model set meet or exceed predefined thresholds for entering the converging phase. Within the converging phase, only selected data is collected and used to retrain the early detection models. The converging phase ends, and the performance phase is entered, when the evaluation metrics of the current early detection models meet or exceed some predefined thresholds for entering the performance phase. During the performance phase, ongoing collection of scalp EEG data allows the early detection model set to be regularly improved. However, If the evaluation metrics of the current early detection model set shows the performance to have declined below the predefined thresholds for entering the performance phase, the seizure early detection system returns to the converging phase.

Determination and refinement of estimations of the duration of the Early State are carried out during the initial and converging phases using, for example, a procedure described below in conjunction with FIG. 6.

FIG. 6 is a flow chart illustrating a procedure for determining the duration of the Early State of an EEG data segment and its corresponding “preictal” annotations. At step 600, when the user's scalp EEG data segment is received into the seizure early detection system, steps 500 to 540 of FIG. 5a are first carried out. Then, if the data segment is not annotated “non-ictal”, steps 570-573 of FIG. 5a are carried out. (In FIG. 6, steps 570-573 in FIG. 5a are represented by step 602.) Otherwise, at decision step 603, the seizure early detection system determines whether the scalp EEG data segment should be annotated “inter-cluster” or “postictal,” by carrying out steps 560-563 of FIG. 5a. If the data segment is annotated neither “inter-cluster” nor “postictal,” steps 564-565 in FIG. 5a are carried out to annotate the data segment “preictal.” Step 564 requires the value for the duration of the Early State, which is obtained according to the procedure in the following, remaining description of FIG. 6.

At step 604, the seizure early detection system determines whether or not it is operating under either the initial phase or the converging phase. If the seizure early detection system is operating under neither phase, i.e., it is operating under the performance phase, duration T_{ESf} of Early State has already been determined. In the performance phase, the seizure early detection system determines, at step 610, the time difference between timestamp T_{nic} of the current “non-ictal” data segment and timestamp T_{ic_f} of the earliest following ictal data segment (i.e., the time to the next ictal data segment, presumably an onset of seizure). This time difference is examined to determine if it is within duration T_{ESf} of the Early State. If so, the current “non-ictal” data segment is annotated as “preictal_{f}” at step 611. The subscript f in the annotation “preictal_{f}” identifies that the annotation is made based on duration T_{ESf} of the Early state and distinguishes this preictal data segment from other preictal data segments that are annotated prior to the determination of duration T_{ESf} of the Early state. Otherwise, i.e., the time to the next ictal data segment is not within the time period T_{ESf} of the Early state, at step 636, the current data segment is annotated “interictal.”

However, at decision step 604, if the seizure early detection system determines that it is in either the initial phase or in the converging phase, the seizure early detection system assigns the data segment to one of several possible values for duration T_{ESi} of the Early state. Specifically, the seizure early detection system may label the data segment “preictalᵢ” where subscript i is an index that identifies the label to be corresponding to which one of several predicted possible durations for the Early state. In one embodiment, the seizure early detection system postulates possible durations T_{ES1}, T_{ES2}, T_{ES5}, T_{ES10}, T_{ES20}, and T_{ES40} for the Early state, corresponding to 1, 2, 5, 10, 20 and 40 minutes prior to the onset of a seizure, respectively. For each data segment, the seizure early detection system determines the time difference between timestamp T_{nic} for the current “non-ictal” data segment under revision and timestamp T_{ic_f} of the earliest next ictal data segment. This time difference is compared, at steps 620-635, against each of durations T_{ES1}, T_{ES2}, T_{ES5}, T_{ES10}, T_{ES20}, and T_{ES40} to determine which of durations T_{ES1}, T_{ES2}, T_{ES5}, T_{ES10}, T_{ES20}, and T_{ES40} is longer than the time difference by the least amount. Of course, the postulated values for the duration of the Early State above are merely illustrative. One may postulate different values, and have a different number of such postulated values, for the duration of the Early state.

The labeled data segments are stored in a buffer in database 530 temporarily. Thereafter, at step 613, representing steps 580-584 of FIG. 5a, their annotations are finalized and stored at step 614 in patient 100's scalp EEG database 172. At step 615, according to the seizure early detection system's scheduling, the early detection models are trained or retrained using the data in database 172. The seizure detection has models Model_{ES1}, Model_{ES2}, Model_{ES5}, Model_{ES10}, Model_{ES20}, and Model_{ES40}, corresponding respectively to postulated durations T_{ES1}, T_{ES2}, T_{ES5}, T_{ES10}, T_{ES20}, and T_{ES40} of the Early state. While the seizure early detection system is still in either the initial phase or the converging phase, as determined at step 640, the seizure early detection system prepares the stored scalp EEG data for training or retraining. Specifically, for each of Model_{ES1}, Model_{ES2}, Model_{ES5}, Model_{ES10}, Model_{ES20}, and Model_{ES40}, the seizure early detection system creates a data set in which data segments are relabeled from “preictal” to either “preictal” or “interictal” based on the following rule:

Preictalᵢ→Preictal if i≤k, and Preictalᵢ→interictal if i>k ∀i, kϵ{1,2,5,10,20,40}
In other words, in the data set associated with Model_{Esk}, each data segment labeled “preictalᵢ” is relabeled “preictal,” if the data segment is originally associated with a duration T_{Esi} that is shorter than duration T_{Esk} of Model_{Esk}, otherwise, the data segment is relabeled “interictal.” With the revised labelling, Model_{ES1}, Model_{ES2}, Model_{ES5}, Model_{ES10}, Model_{ES20}, and Model_{ES40}, respectively, are trained at steps 660-665, using its corresponding relabeled data set.

The training and retraining of Model_{ES1}, Model_{ES2}, Model_{ES5}, Model_{ES10}, Model_{ES20}, and Model_{ES40} allow the seizure early detection system to evaluate each model's performance. The best performing model provides the most reliable prediction in real time of the onset of next seizure episode and thus can be used to provide the most reliable early warning message to the patient. The seizure detection model evaluates performance of each model by statistical measurements of both sensitivity and specificity. In other embodiments, additional or alternative evaluation metrics may be used (e.g., precision and accuracy). From the evaluation, at steps 670 and 672, the seizure early detection system selects one or more eligible models to be used for early detection according to the following rule:

Eligible models={Model_{ESk}|SEN_{ESk}>SEN_{ESthresh} AND SPC_{ESk}≥SPC_{ESthresh}} ∀kϵ{1,2,5,10,20,40}
where SEN_{ESk} and SPC_{ESk} are the sensitivity and specificity of the Model_{ESk} respectively, for kϵ{1, 2, 5, 10, 20, 40}, SEN_{ESthresh} and SPC_{ESthresh} are sensitivity and specificity thresholds for model selection. In other words, a model is eligible if its performances in both sensitivity and specificity surpass respective thresholds.

In one embodiment, sensitivity and specificity thresholds SEN_{ESthresh} and SPC_{ESthresh} are respectively set to 0.8 and 0.7. As a data segment that bears a timestamp closer in time to the onset of a seizure has a greater correlation to the onset of the seizure than a data segment that is not closer, sensitivity and specificity thresholds SEN_{ESk} and SPC_{ESk} may be lesser for models corresponding to longer postulated durations of the Early state. When multiple models are evaluated in accordance with the above eligibility criteria, selecting at step 672 the model corresponding to a longer duration is preferred, to allow an earlier warning to be issued more advance in time. Alternatively, multiple models are maintained to increase reliability of the early detection. If no model is eligible, the model associated with the shortest duration for the Early state (i.e., Model_{ES1}) is selected at step 671. Of course, Model_{ES1} provides the shortest warning time.

To enter the performance phase, the seizure early detection system sets final duration T_{ESf} for the Early state using the following equation:

T_{ESf}=max(T_{ESi}|SEN_{ESi}>SEN_{perf} AND SPC_{ESi}≥SPC_{perf}) if ∃T_{ESi} ∀iϵ{1,2,5,10,20,40}
where SEN_{ESi} and SPC_{ESi} are the sensitivity and specificity of the Model_{ESi} respectively, for iϵ{1, 2, 5, 10, 20, 40}, and SEN_{perf} and SPC_{perf} are sensitivity and specificity thresholds which must be satisfied to enter the performance phase. In other words, to enter the performance phase, final duration T_{ESf} of the Early state is the longest postulated duration T_{ESi} associated with models that have both sensitivity and specificity performances exceeding or equal to the predetermined thresholds SEN_{perf} and SPC_{perf}. It is possible that no such model exists.

When the criterion for selecting final duration T_{ESf} of the Early state is satisfied, the associated model set replaces model set 161 to be used during subsequent operations. The performance phase is entered in the next scheduled retraining of the early detection models. During the performance phase, the seizure early detection system uses final duration T_{ESf} of Early state and, in one embodiment, only a single early detection model need be trained or retrained directly at step 641 according to schedule 615.

Models trained or retrained at step 690 may be stored in model zoo 173, until the next retraining and evaluation at the next scheduled time. The model (i.e., Model_{ESk}) selected during the initial phase or the converging phase, or the model selected during the performance phase becomes the Basic or Refined Early Detection Model at step 673 (as the case may be), which is then converted to model set 161 at step 680 for deployment.

Alternatively, the duration of the Early State may be inferred, for example, using unsupervised machine learning techniques (e.g., as an autoencoder) to generate a mapping of the EEG sequences—which include all the EEG states which the seizure early detection system uses for annotations (e.g., “ictal,” “preictal,” “interictal,”)—into a low-dimensional latent space. Then, by clustering the resulting mapped sequences, one may determine the different EEG states based on the number of individual clusters. Since each sequence in the mapped clusters can be traced back to the exact time in raw EEG data, this clustering technique may determine the optimal preictal starting point.

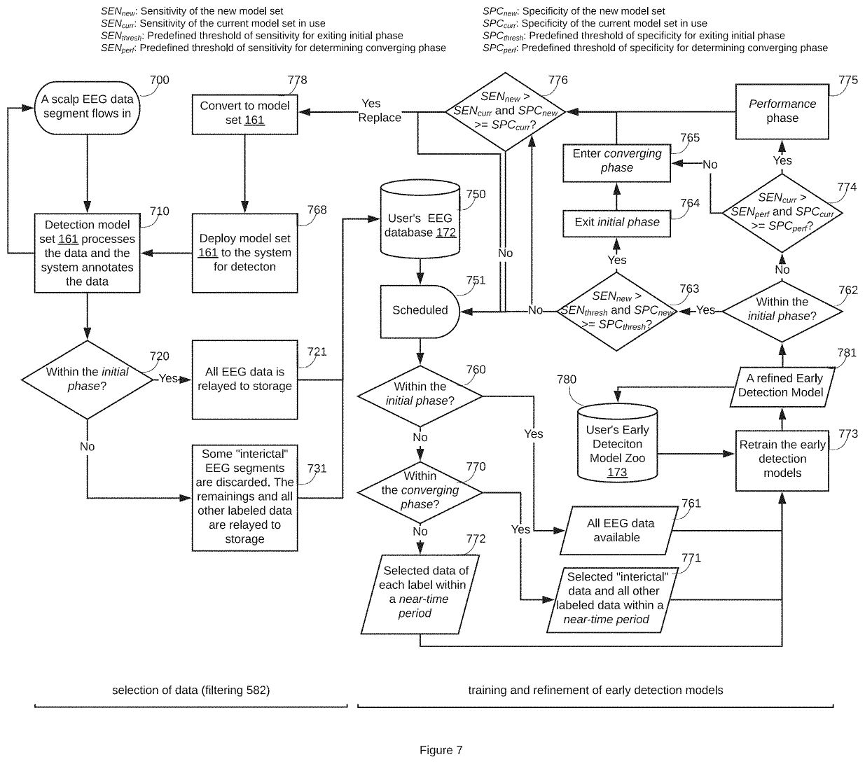

As patient 100's day-to-day scalp EEG data is recorded, annotated and stored in database 172, a continuously updated data set is built up over time for training or retraining the seizure early detection models. FIG. 7 is a flow chart illustrating a procedure in the seizure early detection system for filtering and selecting the collected data for training and retraining.

As mentioned above, transitions among the initial, the converging and the performance phases are based on evaluation of metrics (e.g., the statistical measurements of sensitivity and specificity.) There are other suitable metrics for such evaluation (e.g., precision and accuracy). In one embodiment, as an example, to exit the initial phase, the sensitivity of a retrained model must exceed the predefined sensitivity threshold SENₜₕᵣₑₛₕ of 0.8, and the specificity of that retrained model must exceed the predefined specificity threshold SPCₜₕᵣₑₛₕ, of specificity 0.7. These thresholds are deemed to represent basic performance in the seizure early detection model. Likewise, to exit the converging phase, the sensitivity of a retrained model must exceed the predefined sensitivity threshold SENₜₕᵣₑₛₕ of 0.9, and the specificity of that retrained model must exceed the predefined specificity threshold SPCₜₕᵣₑₛₕ, of specificity 0.8. These thresholds are deemed to represent high performance in the seizure early detection model.

At step 700, scalp EEG data segments are received in real time, processed by the seizure early detection model set 161, and annotated at step 710, as described above in conjunction with FIGS. 5 and 6. If the seizure early detection system is in the initial phase, as determined at decision step 720, received scalp EEG data are relayed at step 721 to storage in patient 100's EEG database 172 at step 750. However, if the seizure early detection system is not in the initial phase, some of the “interictal” EEG data segments may be discarded, saving only a portion of the “interictal” EEG data in database 172 at step 731 All non-interictal scalp EEG data are, however, are replayed and stored in database 172. Discarding interictal data reduces and simplifies the storage space required in database 172. Outside of the initial phase, not all data is required for retraining the early detection model or models. Specifically, in general, the interictal data collected dominate the scalp EEG data collected, which is more than required for model refinement. Interictal data segments may be discarded using a random process, may be selected for discard in batches based on contiguous timestamps within each batch, or selected for discard based on certain times of the day, for example.

As mentioned above, schedule 751 determines when the current seizure early detection models of model set 161 are retrained using the data collected in database 172 and based on which of the initial, converging and performance phases the seizure early detection system is operating under. For example, in one embodiment, during the initial phase, model set 161 is retrained once every three days; during the converging phase, model set 161 is retrained once every two weeks; and, during the performance phase, model set 161 is retrained once every month. In one embodiment, the elapsed time since entering the performance phase may also affect the schedule. In general, the longer the seizure early detection system stays in the performance phase, the less frequent the model need to be retrained. Of course, the above schedule considerations are merely exemplary. In general, during the initial and converging phases, as the performance of the early detection models have not been optimized, more frequent retraining is desirable. For example, during the initial phase, as detection is based on the Basic Early Detection Model, which has various biases, frequent retraining helps to attain an increasingly reliable Refined Early Detection Model is desirable.

The current seizure early detection models—while being deployed as model set 161 in the system for processing data and for making ongoing and incoming seizure detections—are also archived in early detection model zoo 173 at step 780. Model zoo 173 keeps at least a copy of the current models and the most recently retrained models, as well as copies of any retrained models deemed of interest. Keeping such copies may help evaluation of the performance metrics (e.g., sensitivity and specificity) and future retraining.

Returning to FIG. 7, at decision step 760, during the initial phase, all the scalp EEG data in database 172 collected—represented by data 761—is included in a retraining. During the converging phase, only selected interictal data and all other labeled data collected within a “near-time period”—represented by data 771—are including in a retraining. Some examples of the discarding unnecessary interictal data has already been described above. In like vein, one may select the “near-time period” data (e.g., data segments with timestamps in the most recent one or two days) for retraining. It is desirable to use the more recent data. During the performance phase, only selected data of each label—represented by data 772—selected according to a predetermined scheme (e.g., randomly) is used for retraining.

Model set 161 is extracted from model zoo 173 and retrained at step 773 using data 761, 771 or 772, depending on which of the initial phase, the converging phase or the performance phase the seizure early detection system is operating under. The Refined Early Detection Model (i.e., model set 781 in FIG. 7) results from training and retraining. A copy of the Refined Early Detection Model is stored in model zoo 173. In FIG. 7, steps 763-775 illustrate the transitions among the initial phase, the converging phase and the performance phase, according to the evaluation metrics described above. Regardless of which phase the seizure early detection system is operating under, retrained model set 781 replaces and becomes current model set 161 at decision step 776, only if retrained model set 781 yields better evaluation metrics.

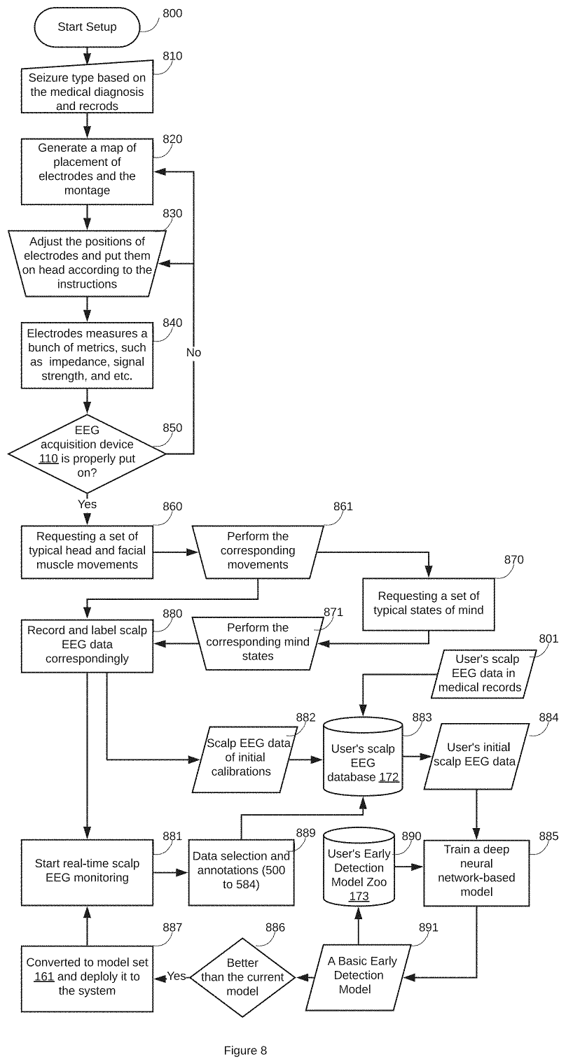

FIG. 8 is a flow chart illustrating a procedure for initializing, calibrating and initially operating acquisition device 110, at patient 100's first use of the seizure early detection system, according to one embodiment of the present invention. At patient 100's initial use of the seizure early detection system (i.e., at step 800), patient 100 provides his or her diagnosed seizure type or/and help obtain his or her medical records from hospitals, other epilepsy facilities, or other relevant sources. The medical records may be uploaded, for example, to patient 100's account in the seizure early detection system at step 810. A map is generated for the electrode placements and montage (i.e., the manner the electrodes are connected). If patient 100's scalp EEG data with seizure onset is available from the previous medical records, the electrode placement map is modified, as necessary, to reflect the original placements at step 820. This electrode placement map may be further modified in the course of use, as necessity.

At step 830, patient 100 places acquisition device 110 on his or her head according to instructions and the electrode placement map, adjusting the positions and the connections of the electrodes of acquisition device 110, as necessary. At step 840, acquisition device 110 takes measurements to establish initial metrics that represents the quality of the scalp EEG data to be collected from the electrodes and provides these metrics to application program 141 running on mobile device 140. The metrics measured may include, for example, impedance, signal strength and other suitable signal quality parameters. At decision step 850, these metrics are used to determine if acquisition device 110 is properly positioned. Patient 100 may have to repeat steps 820-850 one or more times to adjust the position of acquisition device 110 until the measured metrics indicate that acquisition device 110 is operating satisfactorily. The adjustments may include, for example, tuning an amplifier according to the measured signal strength or impedance, tightening the electrodes, generating an alternative map of electrodes placement with better contacts.

After acquisition device 110 is determined to be operating satisfactorily at decision step 850, at steps 860 and 861, patient 100 performs under instruction a set of typical body movements to uncover muscle artifacts in scalp EEG recording. These movements include blinking of the eyes, shaking and turning the head left and right, nodding and moving the head up and down, opening and closing the mouth, smiling and other every day human motions. Concurrently, at step 880, scalp EEG data is collected and labeled during these movements is recorded and labeled. The seizure early detection system also requests patient 100, at steps 870-871, to provide a set of typical states of mind (e.g., closing the eyes to engender a relaxed metal state, focusing on specific objects (e.g., pictures and reading material), or having certain thoughts to engender certain emotions), while concurrently, scalp EEG data is recorded and labeled.

After these initial calibrations, at step 881, the seizure early detection system begins collecting patient 100's scalp EEG and detecting ongoing and incoming seizures, as already described above, using a pre-trained initial model. The scalp EEG data of the initial calibration—indicated in FIG. 8 as data 882—is archived in patient 100's scalp EEG database 172. At step 889, as patient 100's scalp EEG data is being collected, the annotation steps 500-584 of FIG. 5a are also carried out. Selected and annotated data are accordingly stored in database 172. Optionally, patient 100's scalp EEG data from previous medical records—represented in FIG. 8 as data 801—may be imported into database 172. The seizure early detection system then extracts an initial set of scalp EEG data—represented in FIG. 8 as data 884—from database 172. This initial set may contain data 801, if available, and data 882, which is the scalp EEG data collected during the afore-mentioned exercises of muscle artifacts and typical states of mind, in addition to data from step 889.

At step 885, the seizure early detection system retrains the models using data 884 and the current seizure early detection models, which are obtained at step 890 from the pre-trained model set in model zoo 173. The retraining provides a Basic Early Detection Model (i.e., model set 891), which is then stored in model zoo 173 for future evaluation and retraining. Note that the original model set retrieved from model zoo 173 for retraining is also a Basic Early Detection Model that has been deployed for current use. The retrained model set from the original model set remains a Basic Early Detection Model because very limited set of patient 100's scalp EEG data is available for retraining. At decision step 886, evaluation metrics of the retrained model set are compared against evaluation metrics of the original one (i.e., the current model set) to determine if the retained model set provides better performance, according to the procedures described above. If model set 891 is determined to have better performance, model set 891 is converted to, replaces and becomes model set 161 for deployment.

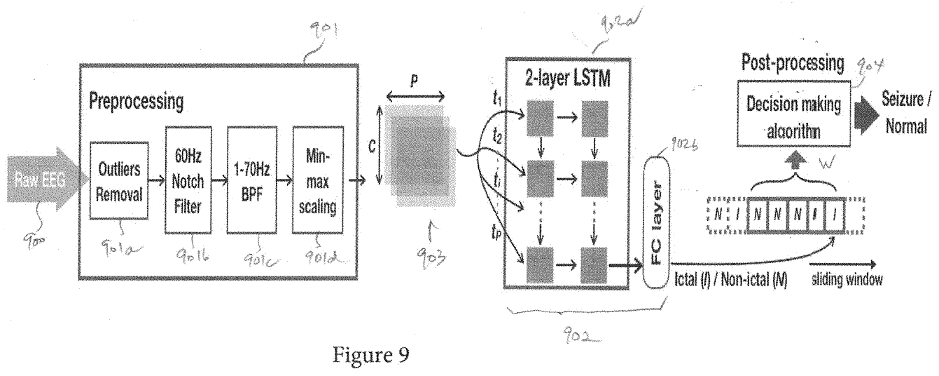

The inventors also recognize that models based on machine learning (e.g., LSTM-based neural networks) can perform seizure detection. FIG. 9 is a block diagram illustrating the operations of a seizure detection system that is driven by a seizure detection machine-learned model designed according to one embodiment of the present invention. In this seizure detection system, scalp EEG data are first pre-processed before processing using a trained deep learning neural network model implemented in a deep-learning neural network (e.g., neural network 902, shown in FIG. 9). The results from the deep-learning neural network are then used to detect a seizure. The inventors have shown that the seizure detection model is effective even with a reduced set of scalp EEG channels (e.g., a small subset of scalp EEG channels selected from the channels under the International 10-20 System).

As shown in FIG. 9, raw scalp EEG data 900 is first preprocessed according to preprocessing steps 901 to eliminate noise and to condition the electrical signal for seizure detection. For example, as shown in FIG. 9, at step 901a, “outlier” signals may be removed. In this context, an outlier signal may be, for example, an unusually large signal fluctuation. As human brain EEG has generally an average peak-to-peak amplitude range of 200.0 μV, any signal that is much greater (e.g. 400.0 μV) may be safely removed as an invalid outlier signal. Further, as shown at step 901b, noise from alternate current (AC) sources (e.g., wall sockets) may be filtered out using a 60-Hz notch filter. The patient's muscles may also be a major source of noise. Such noise, (“muscle artifacts”) may be filtered out by a 1-70 Hz bandpass filter, as indicated at step 901c, since muscle artifacts are typically dominated by of signal components having frequencies higher than 70 Hz. It is also desirable to normalize the signal amplitudes to a desirable range, as shown in step 901d. Normalization step 901d compensates for different settings and for variations among amplifiers in the EEG acquisition devices and allows for variations in absolute EEG signal values measured across patients, or even for variations within the same patient at different measurement times. Normalization may be achieved by min-max scaling to a desirable range of values (e.g., nominally between −1.0 and 1.0). The pre-processing signal treatment steps shown herein are not exhaustive, i.e., many other pre-processing steps for the input EEG signals not shown herein are also advisable.

In some seizure types, rhythmic EEG patterns characteristically appear in certain channels at onset. For example, a focal seizure in the left temporal lobe often first exhibits rhythmic EEG patterns in channels F7-T7 and T7-P7. Therefore, the contemporaneous signals of all measured scalp EEG channels should be analyzed together. Also, epileptic seizure evolves during a seizure episode. For example, the rhythmic EEG activities may start from certain channels and spread to other channels. The amplitudes of the rhythmic signals may also increase during the episode. To capture and use the temporal information, the neural network model should process simultaneously scalp EEG data that are collected over an empirically determined time period. Based on these considerations, returning to the example of FIG. 9, each input vector Xₜ received into neural network 902 labeled with timestamp t has at least two dimensions: a predetermined number of channels (C) and a predetermined number of time points (P). Specifically, input vector Xₜ represents scalp EEG signals from C channels sampled simultaneously at frequency fₛ at P time points over a predetermined duration, p, and P=pfₛ.

As shown in FIG. 9, neural network 902 is formed by combining LSTM neural network 902a with full-connect neural network 902b. LSTM neural network 902a and full-connect neural network 902b may each have a recurrent neural network (RNN) architecture. Specifically, a 2-layer LSTM model may be implemented in LSTM neural network 902a. The inventors believe that the RNN architecture is suitable for capturing the EEG patterns evolution over time. As shown in FIG. 9, input vector Xₜ provides EEG data sampled from the channels at time points t₁, t₂, . . . , tᵢ, . . . tₚ, 1<i≤P, the data from each channel being provided to one corresponding recurrent LSTM iteration at the first layer of LSTM neural network 902a.

The 2-layer LSTM model in LSTM neural network 902a captures the complexity of the dynamic variation of EEG seizure characteristics. In one embodiment, each layer of the 2-layer LSTM model has 256 hidden dimensions. As shown in FIG. 9, the output vector of each recurrent iteration of the first layer of the 2-layer LSTM model is fed into a corresponding recurrent iteration of the second layer, and the output of the last recurrent iteration of the second layer (i.e., the one with the largest delay) is passed through fully-connected neural network 902a to provide a 2×1 vector Yₜ. The two values in vector Yₜ are the bases for predicting Ictal(I) or Non-ictal(N), representing the seizure state and the non-seizure state, respectively.

As seizure EEG signals are typically rare as compared to non-seizure EEG signals, the predictions of “I” (i.e., the seizure state) occur much less frequently than predictions of “N” (i.e., the non-seizure state). This imbalance may make training the neural network model more difficult to converge. Therefore, during training, a sliding window W of duration p may be used to over-sample the “I”-labeled EEG signals. In this over-sampling procedure, there are three parameters: (1) the sliding step size in time (s), (2) the amount of time before over-sampling starts preceding a seizure episode (b), and (3) the amount of time after the oversampling ends following a seizure episode (a). Suppose there is a seizure episode, labeled k, which started at timestamp τₖ and lasted duration Tₖ. Timestamp t of the i-th input vector Xₜ may be given by Equation (1).

$$t = {τ}_{k} - b + is , where 0 \leq i \leq ⌊ \frac{{T}_{k} + a + b}{s} ⌋ \qquad ( 1 )$$

A parameter (R, where 0<R≤1) determines the “I” or “N” labeling for input vector Xₜ. Specifically, label L(Xₜ) is given by Equation (2):

$$L ( {X}_{t} ) = \{ \begin{matrix}I & if {P}_{I} \text{/} P \geq R \\ N & otherwise\end{matrix} \qquad ( 2 )$$

where P_{I} is the number of time points in Xₜ at which the EEG data are deemed to be within a seizure episode. For example, if R=1, an input vector is labeled “I” only if the EEG data at all its constituent time points are deemed to be within a seizure episode. The oversampling is a technique that increases the frequency of “I”-labeled input vector occurrences in the training data by limiting the durations of pre-episode and post-episode data included in the training data to a and b, respectively.

During inference operations (i.e., seizure detection, not training), another parameter (d) indicates the duration of the EEG data to be included in a new input vector to the LSTM model in LSTM neural network 902a, d being less than or equal to p. In the new input vector, dfₛ is the number of time points that contain EEG data that do not overlap with previously presented EEG data to the neural network model. A larger d represents a greater amount of new information being provided to neural network 902, thereby potentially enabling a better decision. On the other hand, however, a larger d also increases the delay before neural network 902 can render a decision, and hence delays an early warning that can be delivered to the patient.

Every duration d, neural network 902 provides a “I” or an “N” decision output. Decision module 904 post-processes the “I” and “N” decision outputs of neural network 902 during the inference or seizure detection phase. Algorithm 1 below sets out the operations of decision module 904. Decision module 904 operates with tunable integer parameters A and E and a decision sliding window W that includes the latest w “I” or “N” decision outputs, w≥max(A, E), from neural network 902. Roughly, as a seizure episode may last continuously from a few seconds to more than a minute, decision module 904 declares a transition into a seizure episode from normal after A consecutive “I” decision outputs have been issued from neural network 902, and declares returning to normal from the seizure episode after E consecutive “N” decision outputs have been issued from neural network 902.

Integer parameters A and E modulate the sensitivity of the system by affecting the frequencies of false positive declarations of seizure conditions and false negative declarations of terminations of seizure episodes. A lesser A sensitizes the system to possible seizures and increases the frequency of warnings of a detected seizure episode being sent (but, equivalently, increases the frequency of false alarms). A greater A also may delay reporting to the patient the onset of a seizure episode. Changing the value of E has a like effect with respect to clearing a seizure episode. A and E may be adjusted empirically in practice according to the experienced accuracy performance of the LSTM model and also according to the user's preference.

Algorithm 1 Seizure Detection Decision Making

Initialize State = Normal.
repeat

if A new entry added to W then

Input: W
if State == Normal then

Take A last entry from W, and assign it to W_{A}
if eᵢ == I, ∀eᵢ ∈ W_{A} then

State = Seizure
Send seizure warning to the user

end if

end if
if State == Seizure then

Take E last entry from W, and assign it to W_{E}
if eᵢ == N, ∀eᵢ ∈ W_{E} then

State = Normal
Clear seizure warning

end if

end if

end if

until EEG streaming stops

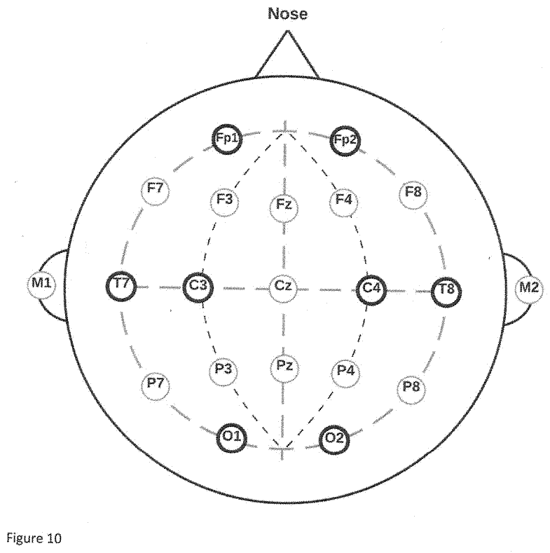

In one embodiment, a reduced set of channels (relative to the International 10-20 system of EEG electrode placements) are used for seizure detection. FIG. 10 indicates the reduced set of channels by circling in bold the implemented electrode placements. Table 1 shows the reduced set of channels (“pseudo-bipolar montage”) created from these electrodes, and the corresponding channels they replace in the standard bipolar montage of the International 10-20 system.

TABLE 1

| | |
|---|---|
| PSEUDO BIPOLAR | STANDARD BIPOLAR |

| | | |
|---|---|---|
| Fp1 - T7 | Fp1 - F7 | F7 - T7 |
| T7 - O1 | T7 - P7 | P7 - O1 |
| Fp1 - C3 | Fp1 - F3 | F3 - C3 |
| C3 - O1 | C3 - P3 | P3 - O1 |
| Fp2 - C4 | Fp2 - F4 | F4 - C4 |
| C4 - O2 | C4 - P4 | P4 - O2 |
| Fp2 - T8 | Fp2 - F8 | F8 - T8 |
| T8 - O2 | T8 - P8 | P8 - O2 |

The set of reduced channels are judiciously selected. While reducing the number of implemented electrodes—and hence the number of channels—used simplifies the EEG acquisition device, the simplification impacts seizure detection. First, the reduced number of electrodes require more time to pick up seizure EEG signals and thus delays the sending of the warning message and reduces the lead time for the patient to react. Second, the reduced number of EEG channels reduce the EEG information fed into the neural network model, thereby making it harder for the model to converge during training.

FIG. 10's electrodes are selected to keep short the delay at an electrode picking up seizure EEG signals at seizure onset. IN FIG. 10, each channel in the pseudo-bipolar montage represents the sum of two corresponding channels in the standard montage. For example, the channel across electrodes at placement points FP1 and T7 (i.e., Fp1-T7) represents the sum of the channels Fp1-F7 and F7-T7 in the standard bipolar montage. Table 1 lists each channel in the pseudo-bipolar montage and its corresponding channels in the standard bipolar montage. Therefore, the pseudo-bipolar montage in some respect compresses the information represented by the standard bipolar montage. The inventors discovered that this pseudo-bipolar montage has not significantly delay the resulting warning message to be sent to the patient, relative to the standard bipolar montage, contrary to expectation.

Transfer learning may be used during training of the neural network model to compensate for the difficulty in model convergence due to the reduced set of channels. Normally, because of the dynamic variations in the EEG signals of different patients, a detection model trained for one patient is not expected to have high accuracy performance when applied on another patient. The inventors discovered, however, that model convergence is in fact helped when weights of one patient's successfully trained model is transferred to initialize the training of another patient's model. With this initialization, convergence was obtained in a model that previously failed to converge. Convergence was attributed to similarities in the EEG patterns between the two patients.

The above detailed description is provided to illustrate specific embodiments of the present invention and is not intended to be limiting. Numerous modifications and variations within the scope of the present invention are possible. The present invention is set forth in the following claims.

We claim:

1. A system for seizure early detection, comprising:
an acquisition device configured to be worn on a patient's head while performing every-day activities, wherein the acquisition device includes electrodes to be positioned at predetermined positions on the patient's scalp for sensing scalp electroencephalogram (EEG) data; and
a mobile device, wherein the mobile device receives the sensed scalp EEG data and provides the sensed scalp EEG data for (i) detecting of an ongoing seizure or (ii) determining a likelihood of occurrence of an upcoming seizure, during the acquisition device's operation, and wherein the likelihood of the upcoming seizure is determined based on a seizure early detection model trained by machine-learning techniques using the sensed EGG data currently collected and in the past.

2. The system of claim 1, wherein the mobile device applies the seizure detection model on the sensed EEG data.

3. The system of claim 1, wherein the acquisition device comprises a transceiver that provides the sensed EEG data over to the mobile device over a wireless connection.

4. The system of claim 1, wherein the mobile device provides the sensed EEG data to a remote computing system on which training of the seizure early detection model takes place over a wide-area computer or communication network.

5. The system of claim 4, wherein the mobile device accesses the wide-area computer or communication network over WiFi.

6. The system of claim 4, wherein the mobile device accesses the wide-area computer or communication network over a cellular telephone network.

7. The system of claim 4, wherein the remote computer system implements the machine-learning techniques in a deep neural network.

8. The system of claim 7, wherein the neural network includes long short term memory (LSTM) components.

9. The system of claim 4, wherein the remote computing system comprises a collection of distributed computing resources.

10. The system of claim 9, wherein the system configures for the patient a virtual separate private cloud to prevent unauthorized access to the sensed EEG data and to preserve privacy.

11. The system of claim 4, wherein the remote computing system divides the sensed scalp EEG data into data segments of predetermined duration and associates each data segment to a clinical state related to epileptic seizure, as the scalp EEG data is received into the remote computing system.

12. The system of claim 11, wherein the data segment has a duration that is less than the patient's average epileptic episode.

13. The system of claim 11, wherein the clinical state is one of: “interictal-cluster,” “ictal,” “ictal-cluster,” “postictal,” “preictal,” or “interictal.”

14. The system of claim 11, wherein the remote computing system trains the seizure early detection model using different approaches based on performance evaluation of the seizure early detection model.

15. The system of claim 14 wherein, in a first phase of operation, the remote computing system provides a basic seizure early detection model using publicly available scalp EEG data relevant to the patient's epilepsy type and the patient's own scalp EEG data collected under non-everyday conditions.

16. The system of claim 15, wherein the scalp EEG data collected under non-everyday conditions is retrieved from previous medical records.

17. The system of claim 16, wherein the basic seizure early detection model incorporates, in one or more calibration steps, scalp EEG data collected during performing a predetermined movement or possessing a predetermined state of mind.

18. The system of claim 17, wherein the predetermined movement comprises one or more of: blinking of the eye, shaking and turning the head left and right, nodding and moving the head up and down, opening and closing the mouth, and smiling.

19. The system of claim 17, wherein the predetermined state of mind comprises one or more of: closing one or both eyes to engender a relaxed mental state, focusing on one or more specific objects, and having one or more specific thoughts to engender related emotions.

20. The system of claim 14, wherein the remote computing system trains and retrains successively improved versions of the basic seizure early detection model until a first set of predetermined performance criteria are met, thereby providing a refined seizure early detection model, whereupon a second phase of operation is entered.

21. The system of claim 14, wherein, during the second phase of operation, the remote computing system trains and retrains successively improved versions of the refined seizure early detection model until a second set of predetermined performance criteria are met, thereby providing a refined seizure early detection model, whereupon a third phase of operation is entered.

22. The system of claim 21, wherein the basic and refined seizure early detection models used in the first, second and third phases of operation are trained and retrained at different respective frequencies.

23. The system of claim 22, wherein the first set of performance criteria or the second set of performance criteria relate to sensitivity and specificity of the seizure early detection model to received scalp EEG data collected during occurrence of an Early state of epileptic seizure.

24. The system of claim 23, wherein the computing system derives a duration of the Early state.

25. The system of claim 24, wherein the computing system derives the duration of the Early state by applying a plurality of postulated durations on multiple postulated seizure early detection models, each created based on one of the postulated durations, and selecting at least one from among the postulated seizure early detection models as the seizure early detection model to be use in determining the likelihood of an upcoming seizure episode.

26. The system of claim 25, wherein the postulated durations for the Early state ranges from less than one minute to longer than one hour.

27. The system of claim 25 wherein, if none of the postulated seizure early detection models meet a third set of performance criteria, the postulated seizure early detection model associated with the least postulated duration is selected as the seizure early detection model to be use in determining the likelihood of an upcoming seizure episode.

28. A system for seizure detection from input vectors representing scalp EEG signals of a patient, comprising:
a pre-processing circuit that processes the input vectors to achieve filtering or conditioning of the scalp EEG signals represented in the input vectors;
a trained neural network, implementing a long short term memory (LSTM) model, that receives the processed input vectors and that provide, for each processed input vector, an indicator that represents whether or not a seizure condition is detected; and
a decision processor that receives the indicators and determines whether or not the indicators indicate a seizure state in the patient.

29. The system of claim 28, wherein the input vector represents EEG signals measured from a predetermined number of channels and a predetermined number of time points.

30. The system of claim 29 wherein, during seizure detection, each input vector includes scalp EEG data for a duration with less than the predetermined number of time points.

31. The system of claim 29, wherein the predetermined number of channels are less than the number of channels in the International 10-20 system.

32. The system of claim 31, wherein the predetermined number of channels are derived from electrodes placed only at Fp1, Fp2, T7, C3, C4, T8, O1 and O2.

33. The system of claim 28, wherein filtering the scalp EEG signals comprises applying a 60-Hz notch filter.

34. The system of claim 28, wherein filtering the scalp EEG signals comprises applying a (1-70)-Hz bandpass filter.

35. The system of claim 28, wherein filtering the scalp EEG signals comprises eliminating from the EEG signals any portion that has an amplitude outside an expected range.

36. The system of claim 35, wherein the expected range is between −200 uV and 200 uV.

37. The system of claim 28, wherein condition the scalp EEG signal comprises normalizes the amplitudes of the scalp EEG signals each to a predetermined range of values.

38. The system of claim 28, wherein the LSTM model comprises two or more layers of hidden dimensions.

39. The system of claim 28, wherein the trained neural network further comprises a fully-connected neural network.

40. The system of claim 28, wherein the trained neural network is trained using an oversampling technique applied on input vectors that represent scalp EEG signals of a seizure state.

41. The system of claim 28, wherein the decision processor, in determining the seizure state of the patient is takes into account a first integer parameter and a second integer parameter representing the number of consecutive indicators representing detection of a seizure condition and the number of consecutive indicators representing other than detection of a seizure condition.
